(19) United States
(12) Patent Application Publication
(10) Pub. No.: US 2015/0352191 A1
(43) Pub. Date: Dec. 10, 2015

(54) TREATMENT OF SKIN DISORDERS

(71) Applicant: University of Dundee, Dundee (GB)

(72) Inventors: Andrew South, Dundee (GB); Stephen Watt, Dundee (GB); Irene Leigh, Dundee (GB)

(21) Appl. No.: 14/379,169

(22) PCT Filed: Feb. 14, 2013
(86) PCT No.: PCT/GB2013/050349

§ 371 Date: Aug. 15, 2014

(30) Foreign Application Priority Data
Feb. 15, 2012 (GB) 1202561.5

(51) Int. Cl. A61K 38/44; A61K 38/39; C07K 14/78; A61K 35/36; G01N 33/573; C12Q 1/68

(52) U.S. Cl. CPC A61K 38/44; G01N 33/573; C12Q 1/6883; C07K 14/78; A61K 35/36; A61K 38/39; G01N 2333/90245; C12Y 114/11004

(57) ABSTRACT

The present invention is based upon the finding that certain conditions, diseases and/or disorders affecting the skin, are associated with reduced expression of an enzyme exhibiting oxidoreductase activity. Accordingly, the invention provides oxidoreductase enzymes and/or genes encoding the same for use in treating or preventing disorders of the skin and method for generating Type VII collagen suitable for use in treating disorders of the skin.

### FIELD OF THE INVENTION

The present invention provides compounds, methods and uses for treating disorders of the skin. The invention further provides methods for producing compounds to be used in the treatment of these skin conditions.

### BACKGROUND OF THE INVENTION

Collagens are large extracellular matrix proteins constituting the primary structural component of the majority, if not all, connective tissues. In addition to providing mechanical resilience and stability in a multi-cellular organism, collagens also play a major role in signalling, having the ability to drastically modify cellular behaviour in both an autocrine and paracrine manner.

Human disease associated with collagen production or processing can manifest in a wide range of phenotypes affecting disparate tissues such as bone and skin. One such disease, recessive dystrophic epidermolysis bullosa (RDEB), is a devastating skin blistering disorder associated with widespread erosions and wounds which heal abnormally leaving scarring and an overall disrupted dermal architecture (1).

RDEB is caused by mutations in the COL7A1 gene (2) which encodes the fibrillar type VII collagen that is the main component of anchoring fibrils, structures which tether the epidermis to the underlying dermis in the skin (3). In addition to the misery of persistent and long term burden of severe blistering patients also face the prospect of terminal cutaneous squamous cell carcinoma with horrific punctuality; more than 80% of patients with the most severe form of RDEB will die from this complication by age 40 (4).

Current methods of treatment have focussed on gene, cell (including stem cell) and protein therapies however, they have not proved to be completely effective. As such, there is a need of new and effective treatments for this debilitating condition.

This invention is based on the finding that cultured keratinocytes derived from patients with RDEB express less PLOD3 than cultured non-RDEB keratinocytes. Moreover, it has been observed that a significant proportion of skin LH3 expression can be found at the basement membrane in normal skin and that this expression is greatly reduced in RDEB patient skin. LH3 expression appears to correlate with type VII collagen expression in vivo and in vitro and it is now shown that LH3 binds type VII collagen and that type VII collagen regulates the expression of LH3 in vitro.

The data presented in this application has wide ranging implications not only for therapeutic strategies being explored for the treatment of RDEB but also for the overall dermal architecture which has been shown to be cancer predisposing in this patient group.

### SUMMARY OF THE INVENTION

The present invention is based upon the finding that certain conditions, diseases and/or disorders affecting the skin, are associated with reduced expression of an enzyme exhibiting oxidoreductase activity.

As such, the invention provides an oxidoreductase enzyme and/or gene encoding the same for use in treating or preventing disorders of the skin. The oxidoreductase enzyme and/or gene encoding the same for use may be formulated as a composition together with an excipient (for example a pharmaceutically acceptable excipient).

The invention may further provide the use of an oxidoreductase enzyme and/or gene encoding the same, in the manufacture of a medicament for the treatment or prevention of disorders of the skin.

The invention further extends to methods of treating or preventing skin disorders, comprising administering a therapeutically effective amount of an oxidoreductase enzyme and/or a gene encoding the same to a subject in need thereof.

The term “oxidoreductase enzyme” encompasses those enzymes collectively referred to as “oxygenase” enzymes. The oxidoreductase enzyme may be lysyl hydroxylase 3 (LH3) encoded by the gene PLOD3 (for: Procollagen-lysine, 2-oxoglutarate 5-dioxygenase 3). As such, references to a gene encoding an oxidoreductase enzyme may embrace the PLOD3 gene.

By way of example, this invention may provide LH3 and/or the PLOD3 gene (or compositions comprising the same) for use in treating or preventing disorders of the skin. The invention may extend to methods and medicaments for treating disorders of the skin, said methods and medicaments exploiting LH3 and/or the PLOD3 gene.

The PLOD3 gene is located on chromosome 7 within the locus designated 7q22. Exemplary PLOD3 and/or LH3 sequences may be accessed using the NCBI reference number: NM_{—}001084.4. Specifically, a reference LH3 sequence may correspond to SEQ ID NO: 1 below.

SEQ ID NO: 1
MTSSGPGPRFLLLLPLLLPPAASASDRPRGRDPVNPEKLLVITVATAETE
GYLRFLRSAEFFNYTVRTLGLGEEWRGGDVARTVGGGQKVRWLKKEMEKY
ADREDMIIMFVDSYDVILAGSPTELLKKFVQSGSRLLFSAESFCWPEWGL
AEQYPEVGTGKRFLNSGGFIGFATTIHQIVRQWKYKDDDDDQLFYTRLYL
DPGLREKLSLNLDHKSRIFQNLNGALDEVVLKFDRNRVRIRNVAYDTLPI
VVHGNGPTKLQLNYLGNYVPNGWTPEGGCGFCNQDRRTLPGGQPPPRVFL
AVFVEQPTPFLPRFLQRLLLLDYPPDRVTLFLHNNEVFHEPHIADSWPQL
QDHFSAVKLVGPEEALSPGEARDMAMDLCRQDPECEFYFSLDADAVLTNL
QTLRILIEENRKVIAPMLSRHGKLWSNFWGALSPDEYYARSEDYVELVQR
KRVGVWNVPYISQAYVIRGDTLRMELPQRDVFSGSDTDPDMAFCKSFRDK
GIFLHLSNQHEFGRLLATSRYDTEHLHPDLWQIFDNPVDWKEQYIHENYS
RALEGEGIVEQPCPDVYWFPLLSEQMCDELVAEMEHYGQWSGGRHEDSRL
AGGYENVPTVDIHMKQVGYEDQWLQLLRTYVGPMTESLFPGYHTKARAVM
NFVVRYRPDEQPSLRPHHDSSTFTLNVALNHKGLDYEGGGCRFLRYDCVI
SSPRKGWALLHPGRLTHYHEGLPTTWGTRYIMVSFVDP

In view of the above, not only does this invention provide oxidoreductase enzymes and genes encoding the same for use in treating disorders of the skin, the invention also provides nucleic acid sequences which encode amino acid sequences exhibiting a degree of homology or identity to a sequence of SEQ ID NO: 1, for use in treating disorders of the skin.

This invention also relates to fragments of the LH3 enzyme and/or fragments of the PLOD3 gene for use in treating or preventing disorders of the skin. The invention also relates to fragments of SEQ ID NO: 1 or nucleic acid sequences which encode said fragments, for use in treating or preventing disorders of the skin. One of skill will appreciate that references to “fragments” of the PLOD3 gene and/or SEQ ID NO: 1, includes fragments which retain oxidoreductase activity. Such “fragments” may also encompass LH3 fragments which retain activity characteristic of the native or complete LH3 enzyme (i.e. LH3 enzyme activity). Similarly, references to fragments of the LH3 enzyme encompass fragments which retain the activity of the native or complete LH3 enzyme.

A fragment of the PLOD3 gene may comprise between about 50 and about n−1 nucleotides of the complete PLOD3 sequence (where “n”=the number of nucleotides in the complete PLOD3 sequence). For example, a PLOD3 fragment encoding a functional LH3 fragment, may comprise 50, 100, 200, 300, 400, 500, 1000, 2000, 3000, 5000, or about 9000 (contiguous) nucleotides of the complete PLOD3 sequence.

A fragment of the LH3 enzyme may comprise between about 10, 50, 100, 200, 300, 400, 500, 600, 700 and n−1 amino acids, wherein “n” is the number of amino acids present in the complete LH3 sequence.

The invention also relates to nucleic acid sequences which exhibit a degree of homology/identity with a reference PLOD3 sequence, such as for example, the exemplary sequence mentioned above. Nucleic acid sequences of this invention may also encode the sequence of SEQ ID NO: 1 or (functional) fragments thereof.

The term “degree of homology/identity” may encompass nucleic acid and/or amino acid sequences which exhibit at least about 30%, 40%, 50%, 60%, 65%, 70%, 75%, 80%, 85%, 90%, 91%, 92%, 93%, 94%, 95%, 96%, 97%, 98% or 99% homology or identify with a PLOD3 sequence or a fragment thereof.

The degree of (or percentage) “homology” between two or more (amino acid or nucleic acid) sequences may be determined by aligning two or more sequences and determining the number of aligned residues which are identical or which are not identical but which differ by redundant nucleotide substitutions (the redundant nucleotide substitution having no effect upon the amino acid encoded by a particular codon, or conservative amino acid substitutions).

A degree (or percentage) “identity” between two or more (amino acid or nucleic acid) sequences may also be determined by aligning the sequences and ascertaining the number of exact residue matches between the aligned sequences and dividing this number by the number of total residues compared—multiplying the resultant figure by 100 would yield the percentage identity between the sequences.

One of skill will appreciate that a nucleic acid sequence which exhibits a degree of homology or identity with another sequence may selectively hybridise or form a duplex therewith. Hybridisation may occur under conditions of high, medium and/or low stringency. Typically, stringent conditions will be those in which the salt concentration is at least about 0.02 molar at pH 7 and the temperature is at least about 60° C. Highly stringent conditions may comprise procedures involving overnight hybridization at about, for example, 68° C. in a 6×SSC solution, washing at room temperature with 6×SSC solution, followed by washing at about, for example, 68° C. in a 6×SSC solution then in a 0.633 SSX solution.

Mutant, variant and/or derivative LH3 and/or PLOD3 sequences are also to be regarded as useful in this invention. A mutant, variant or derivative sequence may, relative to a reference sequence (for example the exemplary PLOD3 or LH3 sequences described herein) comprise one or more nucleotide/amino acid additions, substitutions, deletions and/or inversions. Additionally, or alternatively, a mutant, variant or derivative LH3 and/or PLOD3 sequence may comprise one or more conservative amino acid substitutions. One of skill in this field will understand that a conservative substitution, represents one or more residues which are different from the residues present in a reference sequence, but which do not substantially alter the physico-chemical properties and/or structure or function of the protein.

As is well known in the art, the degeneracy of the genetic code permits substitution of one or more bases in a codon without changing the primary amino acid sequence. Consequently, although the sequences described in this application (for example the exemplary LH3 and/or PLOD3) are known to encode oxidoreductase enzyme is lysyl hydroxylase 3, the degeneracy of the nucleic acid code may be exploited to yield variant nucleic acid sequences which encode the same primary amino acid sequences.

In view of the above, it should be understood that all references to LH3 and/or PLOD3 used herein are to be taken as references to the LH3 enzyme, the gene (PLOD3) encoding the same as well as fragments, variants and/or derivatives thereof.

One of skill will appreciate that the enzyme for use, medicaments or methods of this invention may be administered or applied to subjects suffering from a skin disorder or subjects suspected of, or predisposed to, suffering from a skin disorder. The enzyme for use (or compositions comprising the same), medicaments or methods provided by this invention may be administered or applied prophylactically.

The term “disorders of the skin” may include diseases and/or conditions which affect the integrity of the skin including those characterised by deficiencies in the dermal-epidermal architecture of the skin. By way of example, diseases encompassed within the scope of this disclosure may include diseases caused or contributed to by COL7A1 mutations. Such diseases may include, for example recessive dystrophic epidermolysis bullosa (RDEB) and/or dominant dystrophic epidermolysis bullosa (DDEB).

The disorder of the skin may be RDEB.

In view of the above, this invention provides LH3 and/or the PLOD3 gene (or fragments of either)—or a composition comprising the same, for use in treating or preventing RDEB. The invention may also provide methods and medicaments which exploit LH3 and/or the PLOD3 gene (or fragments of either) in the treatment of RDEB.

Without wishing to be bound by theory, collagen anchoring fibrils are essential to the functional integrity of the dermoepidermal architecture/junction and extend through the basal membrane. Where the anchoring fibrils exhibit abnormal formation and/or are reduced in number or absent, this can result in weak dermoepidermal junctions causing the epidermis to easily separate from the dermis. Consequently, diseases such as RDEB are characterised by dermolytic blister formation in response to minor trauma.

It is known that RDEB is in part caused by the mutations in COL7A1 (the gene encoding collagen type VII) which result in reduced or absent type VII collagen and lead to aberrant anchoring fibril formation at the dermal-epidermal junction. However, while stem cells and type VII collagen delivery have been exploited in the treatment of conditions such as RDEB, they have not proved to be completely effective. The inventors have now discovered that oxygenase enzyme activity may be crucial in the correct formation of type VII collagen molecules, fibrils and anchoring fibrils.

Without wishing to be bound by theory, the inventors suggest that the pathology associated with RDEB is (at least in part) caused or contributed to by the aberrant post-translational modification of type VII collagen.

By enhancing, increasing, augmenting and/or supplementing the expression function and/or activity of LH3 and/or PLOD3 in a subject suffering from a skin disorder such as RDEB or in subjects predisposed or susceptible to skin disorders such as RBEB, it may be possible restore or increase the post-translational events which ensure the correct formation of type VII collagen molecules, fibrils and anchoring fibrils and restore integrity to the dermoepidermal architecture/junction.

In a further aspect, this invention provides a pharmaceutical composition comprising LH3 and/or PLOD3 together with a pharmaceutically acceptable excipient.

The pharmaceutical composition may be formulated for oral, topical and/or parenteral administration. Compositions provided by this invention may be applied directly to parts of the skin exhibiting pathology (lesions) characteristic of a skin disorder.

The oxidoreductase enzymes and/or compositions for use described herein, may be administered together with an existing treatment for a skin disorder. For example, LH3 and/or PLOD3 may be used together with an existing or alternate RDEB therapy. LH3 and/or PLOD3 may be administered to a subject in need thereof together with a cell/stem cell, gene and or protein (for example type VII collagen) based therapy. One of skill will appreciate that LH3 and/or PLOD3 may be administered concurrently with alternate forms of therapy or separately and at different times.

One of skill in this field will be familiar with the processes involved in type VII collagen synthesis; briefly, the first stage requires the translation of alpha-peptide which comprises a central triple-helical (or collagenous) domain flanked by 2 non-collagenous domains (NC1 and NC2). These peptides are post translationally modified by the addition of hydroxyl groups to lysine and proline residues through the actions of lysyl hydroxylases (such as for example, LH3 (encoded by PLOD3)) and prolyl hydroxylases. This step is essential to the formation of cross-links between collagen peptides. Enzymes such as LH3 then glycosylate (galactosidate then glucosidate) the hydroxylysine residues (not the hydroxyproline residues) and three peptides are able to form a triple helix known as pro-collagen.

Once a collagen triple helix has formed it is secreted from the cell. At this stage, two collagen triple helix molecules bind to form an antiparallel dimer, known as a collagen fibril. Again hydroxylysinc status and glycosylation are important factors in the correct formation of the collagen fibrils. From here, bundles of collagen fibrils laterally associate to form the anchoring fibrils essential for dermal-epidermal stability.

Again without being bound to any particular theory, a reduction in PLOD3 and/or LH3 expression, function and/or activity (manifesting as a reduction in lysyl hydroxylation) is likely to affect not only the formation of the type VII collagen triple helix but the subsequent formation of fibrils and anchoring fibrils. Indeed, a reduction in PLOD3 and/or LH3 activity, function and/or expression may lead to the formation of a “loose” triple helix with less cross-links and a functionally impaired collagen.

Beyond this, and again without wishing to be bound by theory, the inventors suggest that reduced PLOD3 and/or LH3 expression, function and/or activity is likely to affect the hydroxylation/glycosylation events crucial to the formation and maintenance of fibrils and anchoring fibrils.

In view of the above, it is essential that the cellular machinery or other systems exploited in the manufacture or production of type VII collagen for therapeutic use, exhibits high levels of PLOD3 and/or LH3 expression, function and/or activity. Where the production of type VII collagen exploits systems which exhibit low/no levels of PLOD3 and/or LH3 expression, the type VII collagen may not be completely functional.

Current methods of generating or synthesising type VII collagen, do not include steps or processes specifically aimed at ensuring the progression of the post-translational modification events crucial to the formation of the type VII collagen triple helix, fibrils and anchoring fibrils.

As such, this invention may extend to methods of producing synthetic or recombinant type VII collagen. A further aspect of this invention provides a method of synthesising or producing type VII collagen, said method comprising contacting or supplementing a system for producing type VII collagen, with an oxidoreductase enzyme of the type described herein.

The system may be a system for the recombinant production of type VII collagen. The system may comprise a cell, for example a keratinocyte.

In a further aspect, this invention may provide type VII collagen prepared by or obtainable by, one or more of the Type VII collagen producing methods systems described herein, which methods and systems may exploit oxidoreductase enzymes, a gene encoding an oxidoreductase enzyme and/or a nucleic acid sequence encoding an amino acid sequence exhibiting a degree of homology or identity with the amino acid SEQ ID NO: 1 or a fragment thereof. One of skill will appreciate that a nucleic acid sequence may be introduced into the methods or systems described herein in the form of a vector (see below).

The invention may provide type VII collagen for use in treating a skin disorder, wherein the type VII collagen has been pre-treated with an oxygenase enzyme. For example, collagen produced for use in treating skin disorders such as RDEB may be contacted with LH3 prior to use. As explained above, by pre-treating or contacting type VII collagen with an oxygenase enzyme (such as LH3) it may be possible to ensure the progression of the post-translational hydroxylation/glycosylation events essential to the formation of collagen fibrils and correct formation/maintenance of anchoring fibrils in vivo.

The invention provides type VII collagen pre-treated with an oxygenase enzyme such as, for example LH3.

In one aspect, this invention provides a vector, for example an expression vector, comprising a nucleic acid sequence encoding PLOD3 or a fragment thereof—in particular, fragments which encode functional LH3 fragments.

In a further aspect, the present invention may provide a cell, transformed with a nucleic acid sequence encoding PLOD3 and/or a fragment thereof—in particular fragments which encode functional LH3 fragments. The cell may be transformed with a vector provided by this invention. The cell may be a mammalian cell, for example a keratinocyte or keratinocyte progenitor cell. A transformed cell of this invention may be provided for use in the treatment of a skin disorder, for example RDEB. Additionally or alternatively, a transformed cell of this invention may find application in methods for producing or synthesising type VII collagen for use in the treatment of skin disorders.

As stated, the present invention is, in part, based on the finding that reduced levels of PLOD3 and/or LH3 expression, function and/or activity are associated with certain skin disorders (or a susceptibility and/or predisposition thereto), including, for example those caused or contributed to by COL7A1—in particular, RDEB and/or DDEB.

As such, this invention may extend to (in vitro) methods of diagnosing RDEB or a predisposition or susceptibility thereto, the method comprising the steps of

(a) providing a sample from a subject;

(b) detecting a level of PLOD3 and/or LH3 in said sample;

wherein reduced levels of PLOD3 and/or LH3 are associated with RDEB.

It should be understood that the phrase “levels of PLOD3 and/or LH3” encompasses levels of PLOD3 and/or LH3 expression—as evidenced by an increase and/or decrease in PLOD3 mRNA/DNA expression or LH3 protein as well as increases and/or decreases in levels of PLOD3 and/or LH3 function and/or activity. A level of LH3 function or activity may manifest as an increase and/or decrease in LH3 enzyme function and/or activity. As such, the term “levels of PLOD3 and/or LH3” includes increases and/or decreases in PLOD3 and/or LH3 expression, function and/or activity.

A sample for use in the method provided by this aspect of this invention may be provided by a subject to be tested for RDEB and/or a predisposition/susceptibility thereto; subjects of this type may exhibit symptoms characteristic of RDEB. The sample may be provided by asymptomatic subjects for the purposes of identifying a predisposition/susceptibility thereto.

A sample for use in this invention may comprise a quantity of protein and/or nucleic acid. As such, the term “sample” should be understood as including samples of bodily fluids such as whole blood, plasma, serum, saliva, sweat and/or semen. In addition, a sample may comprise a tissue or gland secretion and washing protocols may be used to obtain samples of fluid secreted into or onto various tissues, including, for example, the skin. In other instances “samples” such as tissue biopsies and/or scrapings may be used. In particular, cutaneous (i.e. skin) tissue biopsies and/or scrapings may be used. Advantageously such biopsies may comprise keratinocyte cells and in some cases, the keratinocytes and/or biopsy as a whole, may be obtained from tissues exhibiting pathology associated with or indicative of RDEB.

As stated, this invention resides, in part, in the finding that there is reduced PLOD3 and/or LH3 expression, function and/or activity within the basement membrane of the skin of subjects suffering from, disorders such as RDEB. As such, the methods of diagnosing skin disorders (such as, for example, RDEB (or DDEB)) may comprise providing a sample of basement membrane.

Samples subjected to the methods described herein are probed for levels of PLOD3 and/or LH3 (or fragments thereof) and one of skill will appreciate that levels of gene/protein may be assessed relative to a control or reference level the same gene and/or protein.

An increased and/or decreased level of PLOD3 and/or LH3 may be identified by comparing levels of PLOD3 and/or LH3 identified in a sample with a reference or control level of PLOD3 and/or LH3.

Reduced levels of PLOD3 and/or LH3 expression, function and/or activity are associated with instances of RDEB and a reduced level of PLOD3 and/or LH3 may be detected and/or identified in a sample by comparing an identified level of PLOD3 and/or LH3 with a control or reference level of PLOD3 and/or LH3.

There are many ways in which levels of PLOD3 and/or LH3 may be detected and/or identified in samples such as those described herein. By way of example, molecular or PCR based techniques may be used to detect levels of PLOD3 gene expression or gene quantity in a sample. Useful techniques may include, for example, polymerase chain reaction (PCR) using genomic DNA as template or reverse transcriptase (RT)-PCR based techniques in combination with real-time PCR (otherwise known as quantitative PCR). In the present case, real time-PCR may used to determine a level of PLOD3 expression. Typically, and in order to quantify the level of expression of a particular nucleic acid sequence, RT-PCR may be used to reverse transcribe the relevant mRNA to complementary DNA (cDNA). Preferably, the reverse transcriptase protocol may use primers designed to specifically amplify an mRNA sequence of interest (in this case cSCC gene derived mRNA). Thereafter, PCR may be used to amplify the cDNA generated by reverse transcription. Typically, the cDNA is amplified using primers designed to specifically hybridise with a certain sequence and the nucleotides used for PCR may be labelled with fluorescent or radiolabelled compounds.

One of skill in the art will be familiar with the technique of using labelled nucleotides to allow quantification of the amount of DNA produced during a PCR. Briefly, and by way of example, the amount of labelled amplified nucleic acid may be determined by monitoring the amount of incorporated labelled nucleotide during the cycling of the PCR.

Other techniques that may be used to determine the level of cSCC gene expression in a sample include, for example, Northern and/or Southern Blot techniques. A Northern blot may be used to determine the amount of a particular mRNA present in a sample and as such, could be used to determine the amount or level of PLOD3 gene expression. Briefly, mRNA may be extracted from, for example, a sample described herein using techniques known to the skilled artisan. The extracted mRNA may then be subjected to electrophoresis and a nucleic acid probe, designed to hybridise (i.e. complementary) to an mRNA sequence of interest—in this case mRNA encoding PLOD3 gene, may then be used to detect and quantify the amount of a particular mRNA present in a sample.

Additionally, or alternatively, a level of PLOD3 gene expression may be identified by way of microarray analysis. Such a method would involve the use of a nucleic acid probes/primers derived from the PLOD3 gene. Microarrays of this type may be used to identify levels of PLOD3 gene expression, nucleic acid, preferably mRNA, may be extracted from a sample and subjected to an amplification protocol such as, RT-PCR to generate cDNA. Primers specific for sequences encoding the PLOD3 gene may be used.

The amplified (PLOD3) cDNA may be subjected to a further amplification step, optionally in the presence of labelled nucleotides (as described above). Thereafter, the optionally labelled amplified cDNA may be contacted with the microarray under conditions which permit binding with the nucleic acid probes of the microarray. In this way, it may be possible to identify a level of PLOD3 gene expression.

Further information regarding the molecular and PCR based techniques described herein may be found in, for example, PCR Primer: A Laboratory Manual, Second Edition Edited by Carl W. Dieffenbach & Gabriela S. Dveksler; Cold Spring Harbour Laboratory Press and Molecular Cloning: A Laboratory Manual by Joseph Sambrook & David Russell: Cold Spring Harbour Laboratory Press.

In addition, other techniques such as deep sequencing and/or pyrosequencing may be used to detect PLOD3 sequences in any of the samples described above. Further information on these techniques may be found in “Applications of next-generation sequencing technologies in functional genomics”, Olena Morozovaa and Marco A. Marra, Genomics Volume 92, Issue 5, November 2008, Pages 255-264 and “Pyrosequencing sheds light on DNA sequencing”, Ronaghi, Genome Research, Vol. 11, 2001, pages 3-11.

In addition to the molecular detection methods described above, immunological detection techniques such as, for example, enzyme-linked immunosorbent assays (ELISAs) and/or immunohistochemical staining may be used to identify levels of LH3 proteins in samples. ELISPOT, dot blot and/or Western blot techniques may also be used. In this way, samples may be probed for levels of one or more LH3 proteins so as to detect aberrant or modulated expression, function and/or activity which may indicate RDEB or a susceptibility or predisposition thereto.

Further information regarding ELISA procedures and protocols relating to the other immunological techniques described herein may be found in “Using Antibodies: A Laboratory Manual by Harlow & Lane (CSHLP: 1999) and Antibodies: A Laboratory Manual by Harlow & Lane (CSHLP: 1988)”.

Such techniques may require the use of antibodies which exhibit a degree of selectivity, specificity and/or affinity for LH3, fragment(s) and/or epitopes thereof. Antibodies for use in this invention may optionally be conjugated to one or more detectable moieties. By way of example, an antibody for use in any of the immunological detection techniques described herein may be conjugated to an enzyme capable of being detected via a colourmetric/chemiluminescent reaction. Such conjugated enzymes may include but are not limited to Horse radish Peroxidase (HRP) and alkaline phosphatise (AlkP). Additionally, or alternatively, the secondary antibodies may be conjugated to a fluorescent molecule such as, for example, a fluorophore, such as FITC, rhodamine or Texas Red. Other types of detectable moiety include radiolabelled moieties.

The techniques used to generate antibodies are well known in the art and may involve the use of LH3 proteins/peptides (for example LH3 fragments and/or epitopes) in animal immunisation protocols (for the generation of polyclonal antibodies) or as a basis for the generation of hybridomas (for generating monoclonal antibodies). Further information on the preparation and use of polyclonal and/or monoclonal antibodies may be obtained from Using Antibodies: A Laboratory Manual by Harlow & Lane (CSHLP: 1999) and Antibodies: A Laboratory Manual by Harlow & Lane (CSHLP: 1988)—both of which are incorporated herein by reference.

In a further aspect, the present invention provides a kit for use in the detection and/or (in vitro) diagnosis of skin disorder—including, for example RDEB. A kit for use in the diagnosis or detection of a skin disorder may comprise one or more components selected from the group consisting of:
(a) one or more oligonucleotide primers capable of hybridising to sequences of the PLOD3 gene;
(b) one or more antibodies exhibiting specificity, selectivity and/or affinity for LH3 or an epitope thereof;
(c) reagents and/or receptacles for use in methods of diagnosing skin disorders.

The reagents of component (c) may comprise buffers and/or or other solutions (dNTPs, enzymes (polymerase) etc.) for use in the PCR, molecular and/or immunological techniques described herein. Additionally or alternatively, the antibodies of the kit may be conjugated to one or more detectable moieties.

### DETAILED DESCRIPTION

The present invention will now be described in detail with reference to the following figures which show:

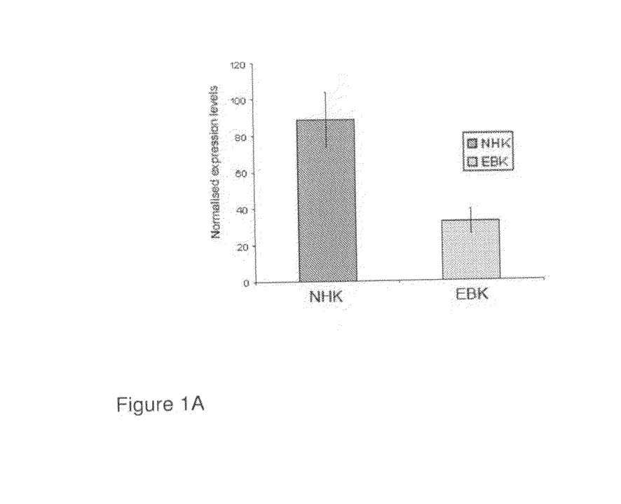

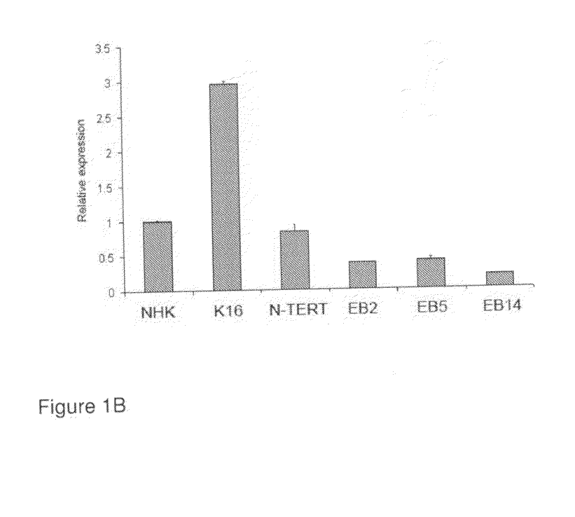

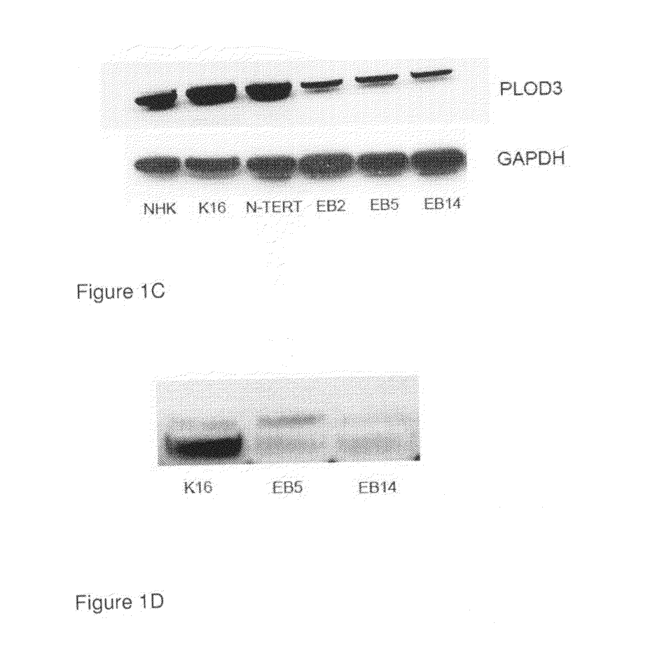

FIG. 1 PLOD3/lysyl hydroxylase 3 is downregulated at both the mRNA and protein level in primary keratinocytes derived from RDEB patients: (A) Microarray analysis comparing cultured keratinocytes derived from RDEB (EBK) and non-RDEB (NHK) patients. Results shown are the mean±SD n=3 and 4 respectively. (B) Quantitative RT-PCR (qRT-PCR) showing higher expression of PLOD3 mRNA in cell lines derived from normal skin (NHK=primary normal human keratinocyte, K16=HPV immortalized normal human keratinocyte, N-TERT=hTERT immortalized human keratinocyte) compared to RDEB derived cells. Results are the mean±SD n=3. Reduced expression (C) and secretion (D) of LH3 protein in RDEB keratinocytes is shown by western blot with anti-PLOD3 antibodies. GAPDH is shown as a loading control.

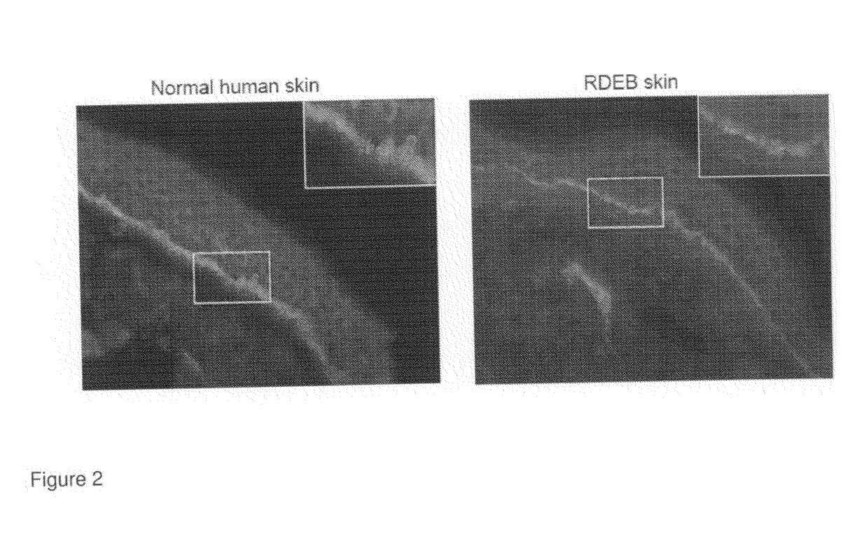

FIG. 2 LH3 localises to the basement membrane in vivo and is reduced in RDEB patients: Immunofluorescence on methanol/acetone fixed frozen sections of both normal human skin (left panel) and RDEB skin (right panel) with PLOD3 specific antibodies shows a strong localization of LH3 at the basement membrane (highlighted and shown in thumbnail) which is substantially reduced in RDEB skin. Staining is also seen in the epidermal keratinocytes.

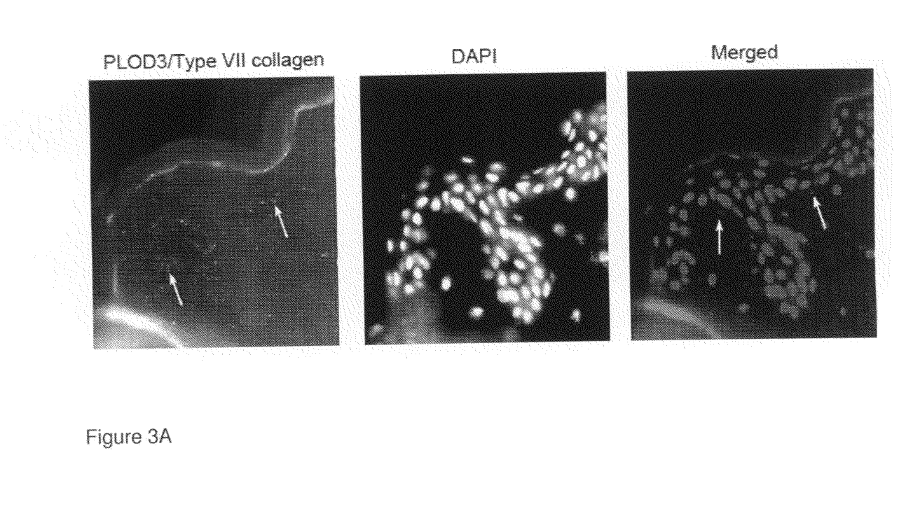

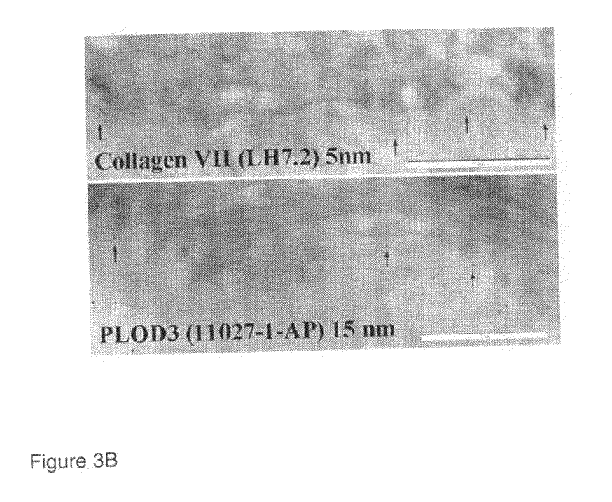

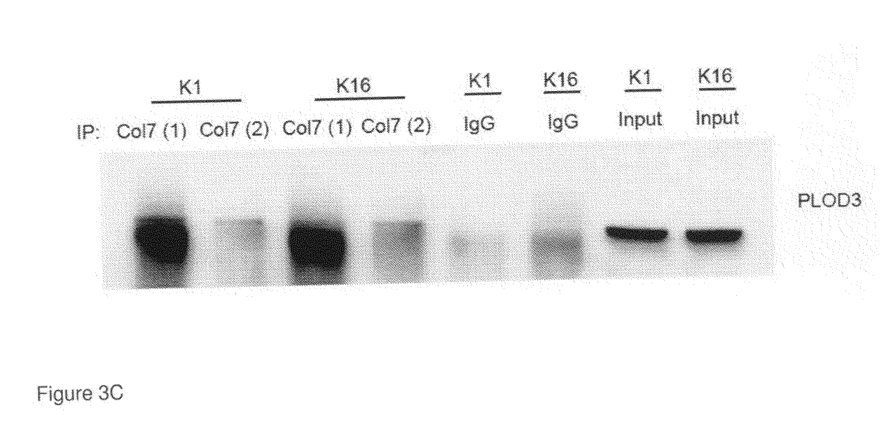

FIG. 3 LH3 and type VII collagen interact at the basement membrane in vivo and in keratinocytes in vitro: (A) Proximity ligation assay (PLA) using antibodies to PLOD3 and type VII collagen indicates interaction of the two proteins along the basement membrane (arrows indicate areas of PLA). (B) Immunogold labelling on ultrathin cryosections of human skin with type VII collagen (upper panel) and PLOD3 (lower panel) antibodies demonstrate localisation of the two proteins to similar areas of the basement membrane. Arrows point to gold particles. (C) Co-immunoprecipitation with polyclonal type VII collagen antibodies (2 antibodies) in whole cell lysates of normal keratinocytes indicate interaction with LH3 in vitro.

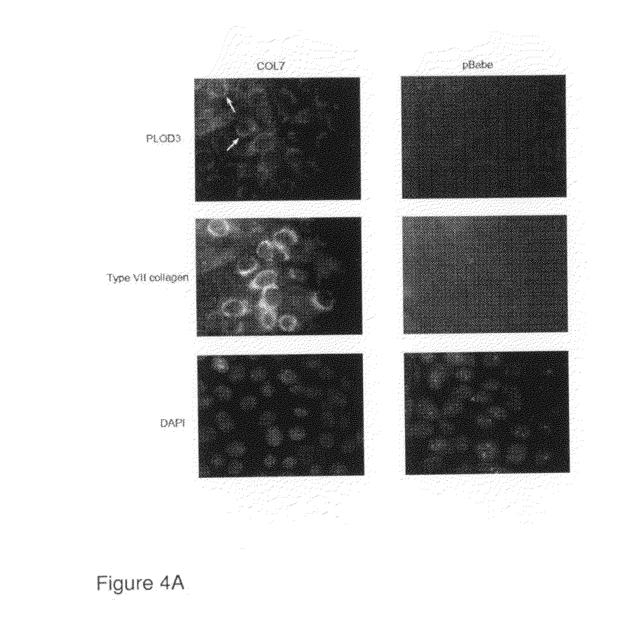

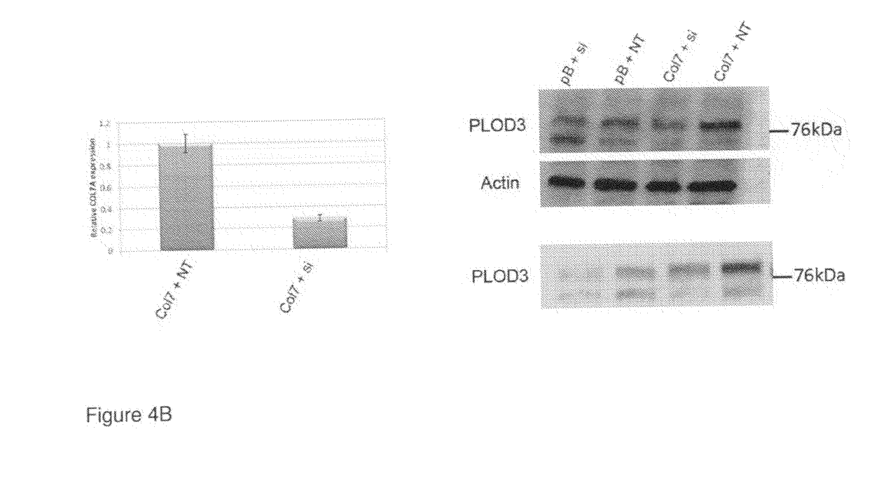

FIG. 4 Expression of type VII collagen influences LH3 levels: (A) EB14 type VII collagen null cells were retrovirally transduced to express type VII collagen (COL7) with the empty vector used as a control (pBabe). Immunofluorescence using antibodies to LH3 (PLOD3) and type VII collagen shows an increase in LH3 expression in the majority of cells which express type VII collagen (arrows indicate examples). (B) Depletion of COL7A1 by siRNA in EB14 COL7 cells, knockdown compared to a non-targeting control siRNA (NT) confirmed by qRT-PCR (left panel), results in a reduction in LH3 protein expression (right panel top, higher bands) and secretion (right panel bottom, higher bands).

### Materials and Methods

All human samples were collected after informed, written consent and in accordance with Helsinki guidelines.

### Keratinocyte Isolation and Culture

Primary keratinocyte cultures were isolated following a standard procedure (29). Briefly, keratinocytes were obtained from biopsies of skin from non-EB and RDEB individuals. After mechanical dissociation, the biopsy fragments were immersed for 1 hour at 37° C. in a trypsin-EDTA solution. Then, the solution was filtered through a 100 μm pore cell strainer (VWR) and medium supplemented with 10% fetal bovine serum was added to neutralized trypsin. Cells were isolated using a centrifuge (5 minutes, 1000 r.p.m.) and the pellet was resuspended in normal keratinocyte medium. Finally, the cells were seeded in T25 flasks containing feeders. The keratinocytes were maintained in DMEM/Ham's F12 medium supplemented with 10% fetal bovine serum, 5 μg/ml transferrin, 0.4 μg/ml hydrocortisone, 10-10 M cholera toxin, 10 ng/ml EGF1, 5 μg/ml insulin, and 2 10-11 M liothyronine. Fresh feeder cells were added to the keratinocytes twice a week. Feeder cells were NIH 3T3 cells treated with mitomycin (7 μg/ml during 3 hours). HpV immortalisation was carried out as described (30).

### Real-Time Quantitative PCR

1 μg RNA was cleaned of genomic contamination and incubated with random primers using the QuantiTect reverse transcriptin kit (Qiagen) to generate cDNA. For quantitative detection of PLOD3 mRNA SYBR green master mix (Qiagen) was used with the following primers: forward 5′-CAGCTCCAGGACCACTTCTC-3′ and reverse 5′-ATGAGGATACGCAGGGTCTG-3′. EF1α primers (forward 5′-GAGAGCTTCTCAGACTATCC-3′ and reverse 5′-GTCCACTGCTTTGATGACAC-3′) were used as an internal control. QIAgility (Qiagen) automated PCR workstation was used to set up PCR samples, reactions performed on the Rotor-Gene Q (Qiagen) and expression calculated by the ΔΔCT method (31).

### Tissue Section Preparation and Immunohistochemistry

Skin tissue was washed immediately in phosphate-buffered saline (PBS) before being embedded in OCT compound (VWR, Lutterworth, UK) and snap-frozen in iso-pentane cooled by liquid nitrogen. Cryosections (6 μm thick) were re-hydrated in PBS for 2 minutes at room temperature before blocking of nonspecific immunoreactive sites with 3% bovine serum albumin in PBS for 20 minutes at 37° C. Sections were incubated in the primary antibodies for 1 hour at 37° C. followed by three 5-minutes washes in PBS. They were then incubated with secondary antibodies; goat anti-mouse Alexa Fluor 488 conjugate and goat antirabbit Alex Fluore 568 conjugate (Molecular probes via Invitrogen, Paisley, UK) along with the nuclear counterstain DAPI (Molecular Probes) for 45 minutes at 37° C. After three 5-minutes washes in PBS, sections were rinsed in water, briefly air-dried and mounted with coverslips. Sections were imaged using Nikon eclipse TE2000-S microscope within 18 hours. As control, tissue sections were processed in parallel without adding primary antibody. No reactivity for secondary antibodies was observed on control histological sections. Primary antibody used was a rabbit polyclonal to LH3 (ProteinTech group, Chicago, Ill.)

### Recombinant Type VII Collagen Expression

COL7A1 was cloned in the retroviral vector pBabe-puro using standard molecular biology techniques. The use of the pBabe-puro retroviral vector (32) and phoenix packaging system (33) to introduce full length COL7A1 was as described′ elsewhere (34) with cells selected using puromycin (1 μg/ml).

### Western Blotting

Keratinocytes were cultured for 2 days post-confluence in keratinocyte serum free medium (Invitrogen) containing EGF and bovine pituitary extract then the conditioned media collected and concentrated using Amicon centrifugal filter units (Millipore, Billerica, Mass.) and whole cell lysates collected using RIPA buffer. Complete mini protease inhibitors (Roche Diagnostics Ltd, West Sussex, UK) were added to both conditioned media and lysates. Proteins were resolved on a 4-12% SDS polyacrilamide gel (Invitrogen) and fractionated proteins transferred to Hybond-ECL™ nitrocellulose transfer membrane (Amersham Biosciences, Little Chalfont, UK). The membrane was blocked with 5% non-fat milk in PBS-tween for 1 hour at room temperature before immunoblotting with primary antibodies to LH3 (ProteinTech group) overnight at 4° C. in blocking buffer. Swine anti-rabbit-horseradish peroxidase conjugated secondary antibody was applied for 1 hour at room temperature and antigen-antibody complexes visualized by enhanced chemiluminescence (Amersham Biosciences), according to the manufacturer's instructions. GAPDH antibody was used as a loading control and protein concentrations were measured using the Bradford assay (Sigma, Pool, UK). Conditioned media loading was normalized according to cell lysate protein concentrations.

### Proximity Ligation Assay

In vivo protein-protein interactions were identified using a rabbit polyclonal antibody to LH3 (ProteinTech group) and a mouse monoclonal antibody LH 7.2 to type VII collagen and applying them in the Duolink H fluorescence assay (Olink Bioscience, Uppsala, Sweden) on frozen tissue sections according to manufacturer's instructions.

### Co-Immunoprecipitation

Whole cell lysate was prepared and co-immunoprecipitation carried out using the Universal Magnetic Co-IP kit (Active Motif, Carlsbad, Calif.) according to manufacturer's instructions. Briefly, 100 μg protein lysate was rocked for 1 hour at 4° C. with 1 μs antibody to type VII collagen before addition of magnetic beads to capture immune-complexes.

### Results

### LH3 Localises to the Basement Membrane and is Reduced in RDEB Skin

To identify gene expression changes in RDEB skin compared to normal we analysed microarray data comparing cultured primary human keratinocytes derived from the two patient groups (15). This revealed 82 in vitro differentially expressed genes one of which, Procollagen-lysine 2-oxoglutarate 5-dioxygenase 3 (PLOD3), showed a 2.75-fold downregulation in RDEB keratinocytes (FIG. 1A). As PLOD3 encodes a known collagen modifying enzyme, lysyl hydroxylase 3 (LH3), with putative roles in collagen synthesis and secretion we selected it for further investigation. Down-regulation at the mRNA level was confirmed by qPCR analysis comparing RDEB keratinocytes against a panel of both primary and immortalised normal keratinocytes, revealing reduced expression ranging from 2.3-16.4-fold (FIG. 1B). In agreement with this data, immunoblotting showed that LH3 protein levels are also markedly decreased in RDEB keratinocytes (FIG. 1C). As LH3 has previously been shown to be secreted from cells we tested whether keratinocytes secreted LH3 and whether this was impaired in RDEB cells. Immunoblotting of conditioned media derived from both normal and RDEB keratinocytes consequently showed that indeed LH3 appears to be abundantly secreted from normal keratinocytes in vitro but is significantly reduced in RDEB cells (FIG. 1D).

To assess LH3 levels in vivo we performed immunofluorescence on frozen sections of both normal and RDEB skin. This showed a distribution of LH3 throughout the epidermis but perhaps unexpectedly revealed a striking localisation along the basement membrane (FIG. 2, left panel). In comparison, this basement membrane localised LH3 was dramatically reduced in RDEB skin where it was found to have a fragmented discontinuous distribution similar to that seen with type VII collagen (FIG. 2 right panel, inset). The distribution of LH3 at the basement membrane is likely to be derived from protein secreted by the keratinocytes and not the dermal fibroblasts as no change in protein expression or secretion was noted comparing normal with RDEB fibroblasts (data not shown). Together this data suggests that LH3 is secreted from keratinocytes and deposited at the basement membrane, but in RDEB skin this is severely reduced.

### LH3 Binds to Type VII Collagen In Vitro and Co-Localises at the Basement Membrane In Vivo

To test whether LH3 interacts with type VII collagen at the basement membrane we used an in situ proximity ligation assay with antibodies to LH3 and type VII collagen to detect proximity between our two epitopes. This approach allowed us to visualize protein-protein interactions in their native state and within fixed tissue. FIG. 3A demonstrates that fluorescent signal was achieved, representing close proximity of our two antibodies (<40 nm), in a pattern predominantly along the basement membrane. At the same time we used immunogold labelling and electron microscopy to study the high resolution localisation of both LH3 and type VII collagen and determine whether they are distributed within the same region of the basement membrane zone. Both type VII collagen and LH3 were found in areas within and beneath the lamina densa (FIG. 3B) and quantification of gold labelling based on distance from the plasma membrane revealed that two LH3 antibodies (polyclonal and monoclonal) map to a region between N- and C-terminal specific type VII collagen antibodies (data not shown). In addition to the basement membrane localised LH3 we wanted to determine whether the LH3 found intracellularly in keratinocytes interacted with type VII collagen where it may be involved in the post-translational modification and synthesis of collagen triple helices. We performed co-immunoprecipitation using specific polyclonal antibodies to pull down type VII collagen and immunoblotted for the presence of LH3. Indeed, when immunoprecipitates were run on a Western blot and probed for detection of LH3 we found clear bands at around 85 Kd not present in our negative control samples, indicating that LH3 can be pulled down in an immune-complex with type VII collagen and therefore the two proteins interact within keratinocytes (FIG. 3C). Together this data shows that LH3 and type VII collagen have close interactions both intracellularly and extracellularly at the basement membrane, indicating possible functional roles for LH3 in type VII collagen synthesis and potentially anchoring fibril formation.

### Type VII Collagen Expression Regulates LH3 Levels In Vitro

As LH3 levels are markedly reduced in RDEB cells expressing no or diminished type VII collagen, we sought to discover whether re-expression of type VII collagen affected LH3 expression. RDEB type VII collagen null cells were retrovirally transduced with type VII collagen (COL7) or the empty vector (pBabe/pB) as a control. Immunofluoresence demonstrates the expression of type VII collagen in COL7 cells (FIG. 4A middle panel) and that the majority of these cells have a markedly increased expression of LH3 (FIG. 4A top panel). Not only do these cells have increased LH3 but LH3 appears to co-localise with type VII collagen in an ER-like pattern within the cytoplasm. An increase in LH3 is also seen in COL7 cells by immunoblotting (FIG. 4B right panel top) and the specificity of the response to type VII collagen expression is clearly seen by siRNA (FIG. 4B). Here we can see that siRNA mediated reduction of COL7A1 in COL7 cells, knockdown confirmed by qPCR (FIG. 4B, left panel), produced a marked reduction of LH3 protein (FIG. 4B, right panel top). In addition, this decrease in protein levels was borne out in levels of secreted protein found in the culture medium (FIG. 4B right panel bottom). Overall these results suggest that expression levels of type VII collagen have a profound effect on the expression of LH3 in keratinocytes.

### Discussion

A critical step in collagen biosynthesis is post-translational modification such as hydroxylation of particular proline (Pro) and lysine (Lys) residues, glycosylation of hydroxylysine (Hyl)2 residues, and the formation of covalent intermolecular cross-links. These events are critical for the correct control of collagen fibrillogenesis (5), cross-linking (6), remodelling (7), and collagen-cell interaction (8), processes integral to normal tissue homeostasis.

Lysyl hydroxylase 3 (LH3), encoded by the PLOD3 gene, is an enzyme that modifies lysine residues in collagens and proteins with collagenous sequences. Unlike other lysyl hydroxylases LH3 is multifunctional and, in addition to hydroxylation activity, possesses galactosyltransferase and glucosyltransferase activities (9). Therefore, LH3 is capable of catalyzing three consecutive reactions required for the formation of unique hydroxylysine-linked carbohydrates, galactosylhydroxylysine and glucosylgalactosyl hydroxylysine. Recent studies have begun to elucidate the precise impact LH3 post-translational modifications have on individual collagens and overall extracellular matrix composition of connective tissues. Mouse knockout studies show that lack of LH3 results in embryonic lethality around day 9.5, widespread disruption to basement membrane formation, dilated endoplasmic reticulum (ER) with collagen aggregates observed both intracellularly and extracellularly (10). Investigation of mice heterozygous for LH3 and a human patient carrying a single PLOD3 mutant allele demonstrate that even a moderate decrease in the amount of intracellular LH3 results in a substantial decrease in LH3 secretion leading to changes in deposition and organization of the ECM (11).

LH3 is described in the lumen of the ER of cells as well as the extracellular space of tissues, in serum and on the surface of cultured cells (12-14) where the enzyme is shown to have activity. Localization to the extracellular space, presumably correlated with secretion, has been shown to be tissue-dependent (13). Data from cell studies suggest that LH3 glycosyltransferase activity can promote cell growth, and LH3 glycosyltransferase activity in the extracellular space is required for cell growth and viability in some tissues (14).

Although RDEB is a monogenic disorder known to be caused primarily by mutations of COL7A1 and the consequent lack of functional anchoring fibrils tethering the epidermis to the dermis, clear differences between keratinocytes derived from RDEB skin and normal skin have so far remained incompletely characterised. This study sought to address this issue by performing transcriptomic analysis on cultured keratinocytes derived from RDEB and non-EB skin.

The discovery of down-regulated levels of PLOD3 encoding the enzyme LH3 was intriguing given its' well characterised role in the post-translational modification, synthesis and secretion of other collagens (16, 17). Although LH3 has been shown to be vital for the formation of basement membranes in epithelial tissues during embryonic development (18), we have demonstrated for the first time that it is heavily distributed at the basement membrane of skin, and importantly, that this basement membrane associated LH3 is severely depleted in the skin of RDEB patients. As we have shown that LH3 levels are depleted in RDEB keratinocytes but not dermal fibroblasts, and that RDEB keratinocytes, but not fibroblasts, secrete greatly reduced amounts of LH3 it would appear that the LH3 found at the basement membrane is derived from the epidermal keratinocytes. We have demonstrated that LH3 interacts with type VII collagen both within the keratinocytes and at the basement membrane which suggests it has a functional role both in type VII collagen triple helix formation (through lysyl hydroxylation and glycosylation) and extracellularly either in collagen dimer formation and/or construction of anchoring fibrils. Why LH3 should be found at the basement membrane is unclear, though despite the fact it is thought that all post-translational modifications on hydroxylysine residues happen prior to triple helix formation, at least for fibrillar collagens, it has been shown that LH3 is able to modify extracellular proteins in their native state (13). One explanation for the presence of LH3 at the basement membrane, and potentially at anchoring fibrils, is that it is required for the maintenance of collagen stability and structure. It has been shown that collagens can undergo a series of microunfolded states where helical sequences melt and refold locally (19-21) allowing access for post-translational modification by LH3 and the proper refolding of the collagen. In addition, unwinding of the triple helix has been shown to occur in the extracellular space due to cleavage by proteolytic enzymes (22, 23). In essence, LH3 may be required at these sites to modify and allow repair of the collagen structure, although at the present time it is unclear whether this would be through lysyl hydroxylation, glycosylation or both.

We have also shown that the expression levels of type VII collagen play a role in the regulation of LH3, indicating that LH3 expression is tightly controlled by the need for it as a post-translational modifier of collagen in keratinocytes. Together our data have potential implications for RDEB therapy. Current approaches strive to replace functional type VII collagen at the basement membrane and include gene therapy (24, 25), cell therapy (26, 27) and protein therapy (28). Protein therapy is still at the pre-clinical stage and although various cell-based therapies have shown promise so far, a major drawback has been the lack of formation of normal anchoring fibrils (27) possibly reducing the integrity and longevity of the patients response. It is possible therefore that a molecule such as LH3 could be vital for the proper formation of the collagen molecules and/or formation and maintenance of anchoring fibrils. In terms of protein therapy, if the system used to produce the recombinant protein lacks adequate levels of LH3 this could have a major effect on the integrity of the triple helix which would further impact collagen dimer and anchoring fibril formation downstream. In addition, it is presently unclear how the injected pro-collagen matures to collagen and subsequently aggregates into anchoring fibrils. If LH3 is required for these processes, but is still reduced in RDEB keratinocytes then simply replacing functional collagen will not be enough to allow proper anchoring fibrils to form. The same is true for cell-based therapies, where although functional type VII collagen appears at the basement membrane, the peptides may not be properly modified and their ability to form anchoring fibrils may be compromised. It is possible therefore that to improve current therapies the restoration of LH3 expression in epidermal keratinocytes and/or at the basement membrane may be necessary through a combinatorial approach.

In addition to its potential effect on current therapeutic approaches in RDEB, the reduced LH3 levels in RDEB skin are likely to have a massive impact on the dermal architecture in these patients. Reduction of LH3 has been shown to cause abnormalities in the overall organisation of the extracellular matrix (11) and its role in the synthesis and secretion of type IV collagen (16) suggest that reduced LH3 levels in RDEB could lead to further deleterious changes to the basement membrane region that exacerbate the effect of type VII collagen loss and may in part contribute to the predisposition to cancer in these patients.

In this study we show a clear reduction in RDEB keratinocytes of an enzyme crucial for the post-translational modification and synthesis of collagens. Through demonstration of its localisation at the basement membrane and its interaction with type VII collagen we suggest that the reduction of LH3 in RDEB may have severe consequences for the dermal architecture in these patients and may have important implications for current methods of RDEB therapy.

### REFERENCES

1. Fine J D, Eady R A, Bauer E A, et al. The classification of inherited epidermolysis bullosa (EB): Report of the Third International Consensus Meeting on Diagnosis and Classification of EB. J Am Acad Dermatol 2008; 58: 931-50.
2. Christiano A M, Greenspan D S, Hoffman G G, et al. A missense mutation in type VII collagen in two affected siblings with recessive dystrophic epidermolysis bullosa. Nat Genet 1993; 4: 62-6.
3. Burgeson R E. Type VII collagen, anchoring fibrils, and epidermolysis bullosa. J Invest Dermatol 1993; 101: 252-5.
4. Fine J D, Johnson L B, Weiner M, Li K P, Suchindran C. Epidermolysis bullosa and the risk of life-threatening cancers: the National EB Registry experience, 1986-2006. J Am Acad Dermatol 2009; 60: 203-11.
5. Batge B, Winter C, Notbohm H, Acil Y, Brinckmann J, Muller P K. Glycosylation of human bone collagen I in relation to lysylhydroxylation and fibril diameter. J Biochem 1997; 122: 109-15.
6. Eyre D R, Glimcher M J. Analysis of a crosslinked peptide from calf bone collagen: evidence that hydroxylysyl glycoside participates in the crosslink. Biochem Biophys Res Commun 1973; 52: 663-71.
7. Segrest J P, Cunningham L W. Variations in human urinary O-hydroxylysyl glycoside levels and their relationship to collagen metabolism. J Clin Invest 1970; 49: 1497-509.
8. Vogel W, Gish G D, Alves F, Pawson T. The discoidin domain receptor tyrosine kinases are activated by collagen. Mol Cell 1997; 1: 13-23.
9. Myllyla R, Wang C, Heikkinen J, et al. Expanding the lysyl hydroxylase toolbox: new insights into the localization and activities of lysyl hydroxylase 3 (LH3). J Cell Physiol 2007; 212: 323-9.
10. Rautavuoma K, Takaluoma K, Sormunen R, Myllyharju J, Kivirikko K I, Soininen R. Premature aggregation of type IV collagen and early lethality in lysyl hydroxylase 3 null mice. Proc Natl Acad Sci USA 2004; 101: 14120-5.
11. Risteli M, Ruotsalainen H, Salo A M, et al. Reduction of lysyl hydroxylase 3 causes deleterious changes in the deposition and organization of extracellular matrix. J Biol Chem 2009; 284: 28204-11.
12. Salo A M, Sipila L, Sormunen R, Ruotsalainen H, Vainio S, Myllyla R. The lysyl hydroxylase isoforms are widely expressed during mouse embryogenesis, but obtain tissue- and cell-specific patterns in the adult. Matrix Biol 2006; 25: 475-83.
13. Salo A M, Wang C, Sipila L, et al. Lysyl hydroxylase 3 (LH3) modifies proteins in the extracellular space, a novel mechanism for matrix remodeling. J Cell Physiol 2006; 207: 644-53.
14. Wang C, Kovanen V, Raudasoja P, Eskelinen S, Pospiech H, Myllyla R. The glycosyltransferase activities of lysyl hydroxylase 3 (LH3) in the extracellular space are important for cell growth and viability. J Cell Mol Med 2009; 13: 508-21.
15. Watt S A, Pourreyron C, Purdie K, et al. Integrative mRNA profiling comparing cultured primary cells with clinical samples reveals PLK1 and C20orf20 as therapeutic targets in cutaneous squamous cell carcinoma. Oncogene 2011; 23 May 2011; doi: 10.1038/onc.2011.180.
16. Sipila L, Ruotsalainen H, Sormunen R, et al. Secretion and assembly of type IV and VI collagens depend on glycosylation of hydroxylysines. J Biol Chem 2007; 282: 33381-8.
17. Sricholpech M, Perdivara I, Nagaoka H, Yokoyama M, Tomer K B, Yamauchi M. Lysyl hydroxylase 3 glucosylates galactosylhydroxylysine residues in type I collagen in osteoblast culture. J Biol Chem; 286: 8846-56.
18. Ruotsalainen H, Sipila L, Vapola M, et al. Glycosylation catalyzed by lysyl hydroxylase 3 is essential for basement membranes. J Cell Sci 2006; 119: 625-35.
19. Kadler K E, Hojima Y, Prockop D J. Assembly of type I collagen fibrils de novo. Between 37 and 41 degrees C. the process is limited by micro-unfolding of monomers. J Biol Chem 1988; 263: 10517-23.
20. Miles C A, Bailey A J. Thermally labile domains in the collagen molecule. Micron 2001; 32: 325-32.
21. Leikina E, Mertts M V, Kuznetsova N, Leikin S. Type I collagen is thermally unstable at body temperature. Proc Natl Acad Sci USA 2002; 99: 1314-8.
22. Federman S, Miller L M, Sagi I. Following matrix metalloproteinases activity near the cell boundary by infrared micro-spectroscopy. Matrix Biol 2002; 21: 567-77.
23. Chung L, Dinakarpandian D, Yoshida N, et al. Collagenase unwinds triple-helical collagen prior to peptide bond hydrolysis. EMBO J 2004; 23: 3020-30.
24. Chen M, O'Toole E A, Muellenhoff M, Medina E, Kasahara N, Woodley D T. Development and characterization of a recombinant truncated type VII collagen “minigene”. Implication for gene therapy of dystrophic epidermolysis bullosa. J Biol Chem 2000; 275: 24429-35.
25. Murauer E M, Gache Y, Gratz I K, et al. Functional correction of type VII collagen expression in dystrophic epidermolysis bullosa. J Invest Dermatol; 131: 74-83.
26. Wong T, Gammon L, Liu L, et al. Potential of fibroblast cell therapy for recessive dystrophic epidermolysis bullosa. J Invest Dermatol 2008; 128: 2179-89.
27. Wagner J E, Ishida-Yamamoto A, McGrath J A, et al. Bone marrow transplantation for recessive dystrophic epidermolysis bullosa. N Engl J Med; 363: 629-39.
28. Remington J, Wang X, Hou Y, et al. Injection of recombinant human type VII collagen corrects the disease phenotype in a murine model of dystrophic epidermolysis bullosa. Mol Ther 2009; 17: 26-33.
29. Rheinwald J G, Green H. Serial cultivation of strains of human epidermal keratinocytes: the formation of keratinizing colonies from single cells. Cell 1975; 6: 331-43.
30. Halbert C L, Demers G W, Galloway D A. The E7 gene of human papillomavirus type 16 is sufficient for immortalization of human epithelial cells. J Virol 1991; 65: 473-8.
31. Livak K J, Schmittgen T D. Analysis of relative gene expression data using real-time quantitative PCR and the 2(-Delta Delta C(T)) Method. Methods 2001; 25: 402-8.
32. Morgenstern J P, Land H. Advanced mammalian gene transfer: high titre retroviral vectors with multiple drug selection markers and a complementary helper-free packaging cell line. Nucleic Acids Res 1990; 18: 3587-96.
33. Kinsella T M, Nolan G P. Episomal vectors rapidly and stably produce high-titer recombinant retrovirus. Hum Gene Ther 1996; 7: 1405-13.
34. South A P, Wan H, Stone M G, et al. Lack of plakophilin 1 increases keratinocyte migration and reduces desmosome stability. J Cell Sci 2003; 116: 3303-14.

1-25. (canceled)

26. A method of treating or preventing disorders of the skin, said method comprising administering to a subject in need thereof a therapeutically effective amount of a:
(i) oxidoreductase enzyme;
(ii) gene encoding an oxidoreductase enzyme; and/or
(iii) nucleic acid sequence encoding an amino acid sequence exhibiting a degree of homology or identity with the amino acid sequence of SEQ ID NO: 1 or a fragment thereof.

27. The method of claim 26, wherein the enzyme is lysyl hydroxylase 3 (LH3).

28. The method of claim 26, wherein the gene is the Procollagen-lysine,2-oxoglutarate 5-dioxygenase 3 (PLOD3) gene.

29. The method of claim 26, wherein a disorder of the skin is a disease and/or condition affecting the integrity of the skin and/or characterised by deficiencies in the dermal-epidermal architecture of the skin.

30. The method of claim 26, wherein the disorder of the skin is a disease caused or contributed to by one or more mutations in the COL7A1 gene.

31. The method of claim 26, wherein the disorder of the skin is dystrophic epidermolysis bullosa (RDEB) and/or dominant dystrophic epidermolysis bullosa (DDEB).

32. A pharmaceutical composition comprising LH3 and/or PLOD3 or a fragment, derivative or variant thereof, together with a pharmaceutically acceptable excipient.

33. A method of producing type VII collagen, said method comprising contacting or supplementing a system for producing type VII collagen, with an oxidoreductase enzyme.

34. The method of claim 33, wherein the oxidoreductase enzyme is lysyl hydroxylase 3 (LH3).

35. The method of claim 34, wherein the lysyl hydroxylase 3 (LH3) is encoded by an amino acid sequence having a degree of homology or identity to the sequence of SEQ ID NO: 1.

36. The method of claim 33, wherein the system for producing type VII collagen is a system for the recombinant production of type VII collagen.

37. A method of treating a skin disorder, said method comprising administering to a subject in need thereof, a therapeutically effective amount of a Type VII collagen, wherein the type VII collagen has been pre-treated with an oxidoreductase enzyme.

38. The method of claim 37, wherein the type VII collagen is pre-treated with lysyl hydroxylase 3 (LH3).

39. A vector comprising a nucleic acid sequence encoding an oxidoreductase enzyme.

40. The vector of claim 39, wherein the oxidoreductase enzyme is lysyl hydroxylase 3 (LH3).

41. The vector of claim 39, wherein the nucleic acid sequence exhibits a degree of homology and/or identity to (i) the sequence of the PLOD3 gene or a fragment thereof and/or (ii) a nucleic acid sequence which encodes and amino acid sequence of SEQ ID NO: 1 or a fragment thereof.

42. An isolated cell, transformed with a nucleic acid sequence exhibiting a degree of homology or identity to the sequence of the PLOD3 gene and/or a fragment thereof

43. An isolated cell transformed with the vector of claim 39.

44. The cell of claim 42, wherein the cell is a mammalian cell, a keratinocyte or keratinocyte progenitor cell.

45. The cell of claim 43, wherein the cell is a mammalian cell, a keratinocyte or keratinocyte progenitor cell.

46. A method of treating a skin disorder, said method comprising administering a subject in need thereof a cell of claim 42.

47. A method of treating a skin disorder, said method comprising administering a subject in need thereof a cell of claim 43.

48. A method of producing or synthesising type VII collagen, said method comprising the vector of claim 39.

49. A method of producing or synthesising type VII collagen, said method comprising the cell of claim 42.

50. A method of producing or synthesising type VII collagen, said method comprising the cell of claim 43.

51. A method of diagnosing RDEB or a predisposition or susceptibility thereto, the method comprising the steps of
(a) providing a sample from a subject;
(b) detecting a level of PLOD3 and/or LH3 in said sample;
wherein reduced levels of PLOD3 and/or LH3 are associated with RDEB.

52. The method of claim 52, wherein the level of PLOD3 and/or LH3 is detected by immunological and/or molecular detection techniques.

53. A kit for use in the detection and/or diagnosis of a skin disorder, said kit comprising one or more components selected from the group consisting of:
(a) one or more oligonucleotide primers capable of hybridising to sequences of the PLOD3 gene; and
(b) one or more antibodies exhibiting specificity, selectivity and/or affinity for LH3 or an epitope thereof;

54. Type VII collagen obtainable by the method of claim 33.


SEQUENCE LISTING (5 sequences)

SEQ ID NO: 1 - protein, 738 aa, Homo sapiens
MTSSGPGPRFLLLLPLLLPPAASASDRPRGRDPVNPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRIRNVAYDTLPIVVHGNGPTKLQLNYLGNYVPNGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLATSRYDTEHLHPDLWQIFDNPVDWKEQYIHENYSRALEGEGIVEQPCPDVYWFPLLSEQMCDELVAEMEHYGQWSGGRHEDSRLAGGYENVPTVDIHMKQVGYEDQWLQLLRTYVGPMTESLFPGYHTKARAVMNFVVRYRPDEQPSLRPHHDSSTFTLNVALNHKGLDYEGGGCRFLRYDCVISSPRKGWALLHPGRLTHYHEGLPTTWGTRYIMVSFVDP

SEQ ID NO: 2 - DNA, 20 bp, Artificial
  Primer
cagctccaggaccacttctc

SEQ ID NO: 3 - DNA, 20 bp, Artificial
  Primer
atgaggatacgcagggtctg

SEQ ID NO: 4 - DNA, 20 bp, Artificial
  Primer
gagagcttctcagactatcc

SEQ ID NO: 5 - DNA, 20 bp, Artificial
  Primer
gtccactgctttgatgacac